(19) United States
(12) Patent Application Publication
(10) Pub. No.: US 2002/0160427 A1
(43) Pub. Date: Oct. 31, 2002

(54) Methods and apparatuses for electronic determination of analytes

(75) Inventors: Markus Beier, Heidelberg (DE); Cord F. Stahler, Weinheim (DE)

Correspondence Address: ARENT FOX KINTNER PLOTKIN & KAHN, PLLC, Suite 600, 1050 Connecticut Avenue, N.W., Washington, DC, 20036-5339, US

(73) Assignee: febit ag

(21) Appl. No.: 10/132,167

(22) Filed: Apr. 26, 2002

(30) Foreign Application Priority Data
Apr. 27, 2001 (DE) 101 20 663.1
Nov. 16, 2001 (DE) 101 56 433.3

(51) Int. Cl. C12Q 1/68; G01N 33/53; G01N 33/542; C12M 1/34

(52) U.S. Cl. 435/7.9; 435/6; 435/287.2

(57) ABSTRACT

The invention relates to a method and an apparatus for determining analytes by electronic detection using a microfluidic support.

### DESCRIPTION

The invention relates to a method and an apparatus for determining analytes by electronic detection using a microfluidic support.

In recent years, the technology of receptor arrays immobilized on a support, for example DNA chips, has established a valuable means which enables complex analyte determination methods to be carried out rapidly and in a highly parallel manner. The biophysical principle on which the receptor arrays are based is that of the interaction of a specific immobilized receptor with an analyte present in a liquid phase, for example via nucleic acid hybridization, the support being provided with a multiplicity of receptors, for example hybridization probes, which bind specifically to analytes present in the sample, for example complementary nucleic acid analytes.

A binding event between immobilized receptor and analyte is usually detected via detection of a marker group which is bound to the analyte. A support and a method for analyte determination, which allow an integrated synthesis of receptors and analysis, are described, for example, in WO 00/13018. However, such supports and methods have the disadvantage that binding of analytes without marker group to the receptor cannot be readily detected.

DE 199 01 761, DE 199 21 940 and DE 199 26 457 relate to methods for the electrochemical or electronic detection of nucleic acid hybridization events. In this connection, single-stranded hybridization probes whose one end is bound to a support surface and whose other, free end is linked to a redox active unit serve as hybridization matrix. Hybridization of a nucleic acid analyte increases the originally nonexistent or only weak electric communication between the conductive surface area of the support and the redox active unit. Thus it is possible to detect a hybridization event by electrochemical methods such as voltammetry, amperometry or conductivity measurement. In this connection, photo-inducible or chemically inducible redox active units may be used.

Further methods for electrochemical or electronic detection of hybridization events are described in WO 93/20230 WO 95/12808 WO 97/41425 WO 98/30893, WO 98/51819 WO 00/11473 WO 99/37819 WO 96/40712, U.S. Pat. No. 5,968,745, U.S. Pat. No. 5,952,172 and JP-A-92 88 080.

It was the object of the present invention to provide an integrated system which allows highly parallel in situ preparation of complex populations of receptors, immobilized in microstructures, for the detection of analytes.

The present invention therefore relates to a method for determining analytes, which comprises the following steps:

(a) providing an apparatus comprising

(i) a light source matrix,

(ii) a microfluidic support having channels which contain a plurality of predetermined areas at which in each case different receptors are immobilized on the support,

(iii) means for supplying fluids to the support and for discharging fluids from the support and

(iv) an electronic detection matrix having a plurality of electrodes assigned to the predetermined areas containing immobilized receptors on the support,

(b) contacting the support with a sample containing analytes and

(c) determining the analytes by electronic detection via binding thereof to the receptors immobilized on the support.

The invention further relates to an apparatus for determining analytes, which comprises

(i) a light source matrix,

(ii) a support having channels which contain a plurality of predetermined areas at which in each case different receptors are immobilized on the support,

(iii) means for supplying fluids to the support and for discharging fluids from the support and

(iv) an electronic detection matrix having a plurality of electrodes assigned to the predetermined areas containing immobilized receptors on the support.

The present invention is distinguished in particular by the fact that the detection system for analyte determination combines a light source matrix, a microfluidic support and an electronic detection matrix in an at least partly integrated structure. Said detection system may be used for integrated synthesis and analysis, in particular for the construction of complex supports, for example biochips, and for the analysis of complex samples, for example for genome, gene expression or proteome analysis.

In a particularly preferred embodiment, the receptors are synthesized in situ on the support, for example by directing fluid containing receptor synthesis building blocks over the support, immobilizing said building blocks location- or/and time-specifically at in each case predetermined areas on the support and repeating these steps until the desired receptors have been synthesized at the in each case predetermined areas on the support. Said receptor synthesis preferably comprises at least one illumination step initiated by the light source matrix or/and a process step mediated by the electronic detection matrix and also on-line process monitoring, for example by using the electronic detection matrix. It is possible here to use for the receptor synthesis electronically removable protective groups such as, for example, p-nitrobenzyloxycarbonyl, 2-(p-nitrophenyl)ethyloxycarbonyl, 2,4-dinitrobenzyl oxycarbonyl or/and 2,4(p-dinitrophenyl)ethyl oxycarbonyl.

The light source matrix is preferably a programmable light source matrix, for example selected from the group consisting of a light valve matrix, a mirror array, a UV-laser array and a UV-LED (diode) array.

The support is a flow cell or a microflow cell, i.e. a microfluidic support having channels, preferably closed channels, which contain the predetermined positions with the in each case differently immobilized receptors. The channels preferably have diameters in the range from 10 to 10,000 μm, particularly preferably from 50 to 250 μm, and may in principle be designed in any form, for example having round, oval, square or rectangular cross sections.

The electronic detection matrix contains a plurality of electrodes which are assigned to those areas of the support on which receptors are immobilized. Preference is given to assigning to an area with in each case identical receptors a separate electrode which may be surrounded, for example, by an insulator area. The electrodes of the electronic detection matrix contain a conductive material such as, for example, a metal, for example silicon, a conductive polymer or a conductive glass. The electrodes preferably form an integral part of the microfluidic support and may form, for example, part of the walls of the microchannels of the support. Furthermore, the support is preferably at least partly optically transparent, in particular on the side facing the light source matrix. However, it is not necessary for the support to be optically transparent on both sides. The electrode areas are preferably in the range from 15 to 250,000 μm², particularly preferably in the range from 15 to 2,500 μm².

Electronic detection may be carried out according to known techniques (see, for example, the abovementioned documents), for example by measuring parameters which change in a detectable manner, owing to binding of an analyte to the receptor. Examples of such parameters are conductivity, impedance, voltage or/and current, all of which can be determined via the electrodes using a suitable electronic detector. Depending on the structure of the analytical apparatus, the measurement may comprise a potentiometric measurement, a cyclovoltametric measurement, an amperometric measurement, a chronopotentiometric measurement or another suitable principle of measurement.

In a particularly preferred embodiment, the detection comprises a light source matrix-initiated redox process which correlates with the binding of analytes, for example by hybridization, to the receptors immobilized on the support.

The receptors are preferably selected from biopolymers which may be synthesized in situ on the support from the appropriate synthesis building blocks by light-controlled or/and chemical processes, for example nucleic acids such as DNA, RNA, nucleic acid analogs such as peptide nucleic acids (PNA), proteins, peptides and carbohydrates. Particular preference is given to selecting the receptors from the group consisting of nucleic acids and nucleic acid analogs, and binding of the analytes comprises a hybridization.

The analyte determination of the invention preferably comprises parallel determination of a plurality of analytes, i.e. a support is provided which contains a plurality of different receptors which can react with in each case different analytes in a single sample. Preference is given to the method of the invention determining at least 50, preferably at least 100 and particularly preferably at least 200, analytes in parallel.

The receptors may be immobilized to the support by covalent binding, noncovalent self assembly, charge interaction or combinations thereof. Covalent binding preferably comprises providing a support surface having a chemically reactive group to which the starting building blocks for receptor synthesis can be bound, preferably via a spacer or linker. Noncovalent self assembly may take place, for example, on a noble metal surface, for example a gold surface, by means of thiol groups, preferably via a spacer or linker.

The apparatus of the invention may be used for the electronically controlled in situ synthesis of nucleic acids, for example DNA/RNA oligomers, it being possible to use as temporary protective groups electronically removable protective groups such as, for example, p-nitrobenzyloxycarbonyl, 2-(p-nitrophenyl)ethyloxy carbonyl, 2,4-dinitrobenzyloxycarbonyl or/and 2,4-(p-dinitrophenyl)ethyloxycarbonyl. It is also possible, where appropriate, to use combinations of photoactivatable protective groups, chemical protective groups or/and electronic protective groups. The location- or/and time-resolved receptor synthesis may be carried out by specifically addressing the electrodes of the detection matrix, by specifically supplying fluids to defined areas or area groups on the support or/and by specific illumination via the light source matrix.

The present invention makes possible considerable improvements compared with known analyte determination methods, for example by providing an integrated electronic system for receptor synthesis and for analyte detection without movable parts. The detection may be varied via different designs of the electrode structures. An improved on-line process control may also be achieved by combining light, fluid supply and electronic detection.

Furthermore, the following figures are intended to illustrate the present invention:

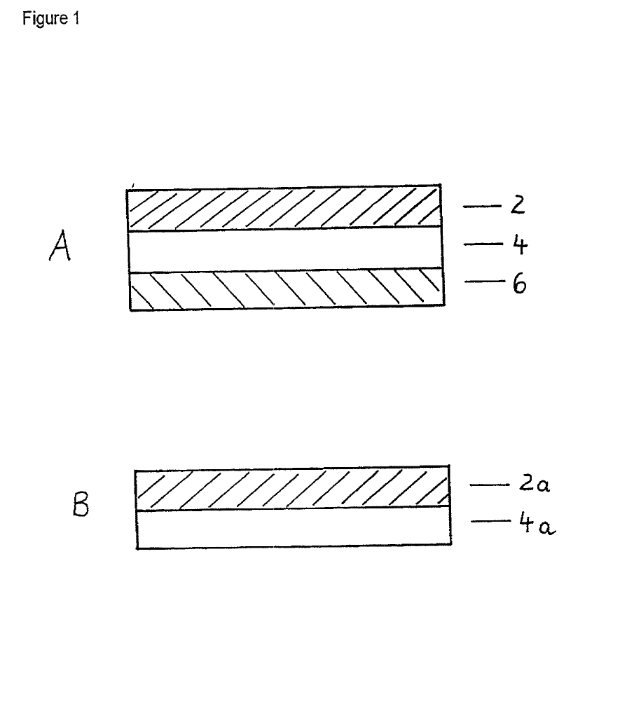

FIG. 1 shows the basic structure of an electronic integrated synthesis-analysis (eISA) system. The system shown in FIG. 1A contains 3 layers, a light source matrix (2), a microfluidic support (4) and an electronic detection matrix (6). The apparatus shown in FIG. 1B consists of two layers, namely the light source matrix (2a) and a microfluidic support with integrated electronic detection matrix (4a).

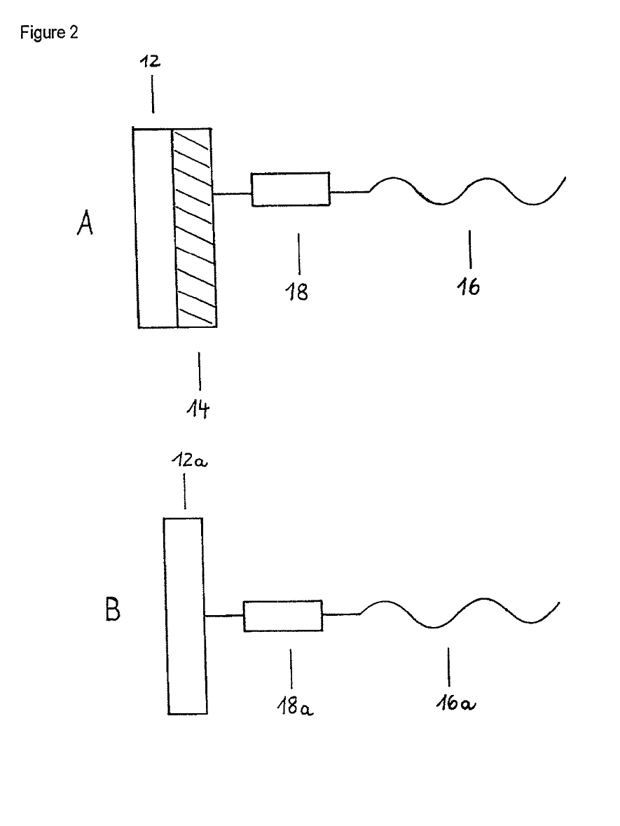

FIG. 2 shows different embodiments for immobilizing receptors, for example a DNA oligomer strand, on the electrode structure. According to FIG. 2A, an electrically conducting layer (12) and above it a permeation layer (14) are provided, to which the receptor, for example a DNA oligomer (16), is bound covalently or noncovalently via a spacer (18). According to FIG. 2B, the receptor (16a) is directly bound covalently or noncovalently via a spacer (18a) to the electrically conducting layer (12a).

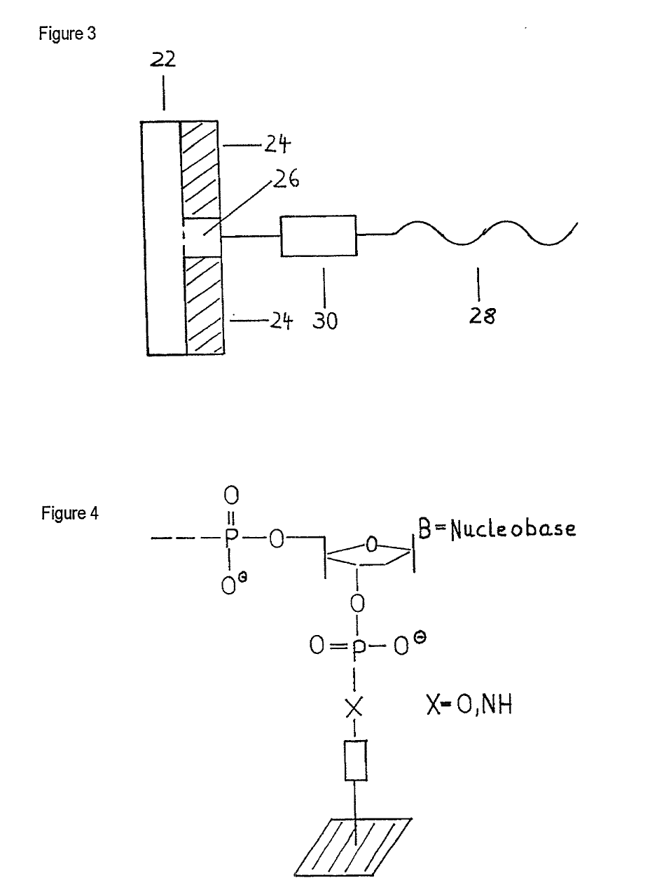

According to FIG. 3, the receptor is bound directly on the electrically conductive layer (22). The surface of the microfluidic support alternately comprises insulating (24) and electrically conductive (26) areas, with the receptor (28) being bound to an electrically conducting area via a spacer (30).

FIG. 4 is a detailed representation of the binding of a DNA oligonucleotide strand to an electrically conductive area (electrode) of the support via a spacer.

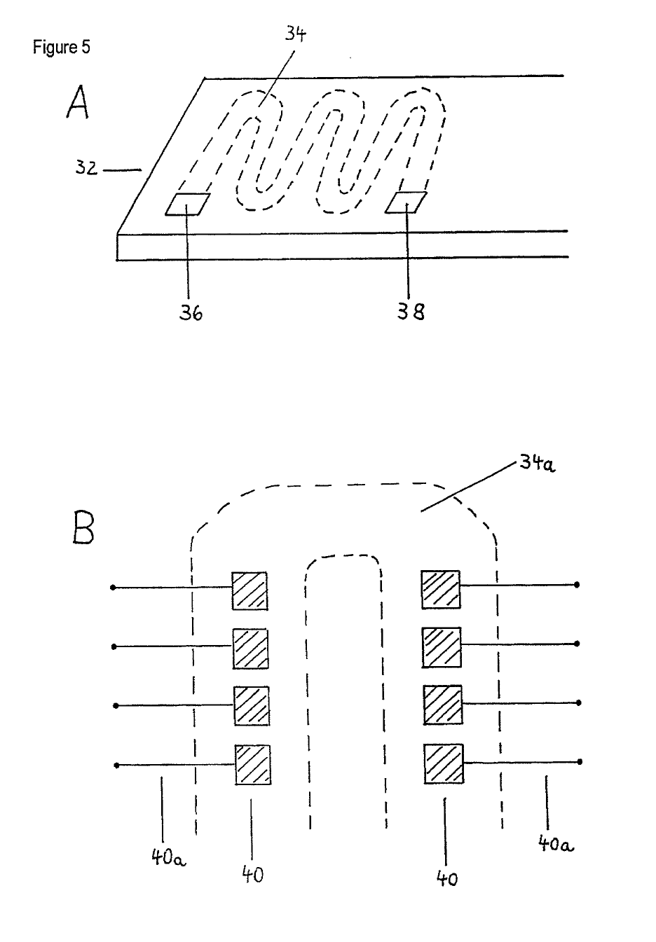

FIG. 5A shows a microfluidic reaction support (32) with a microchannel (34) in the interior of the support and inlet orifices (36) and outlet orifices (38) for fluid. FIG. 5B shows the pattern of an electrode structure (40) with electrically conductive connections (40a ) in connection with a section of the channel structure (34a) of the support shown in FIG. 5A.

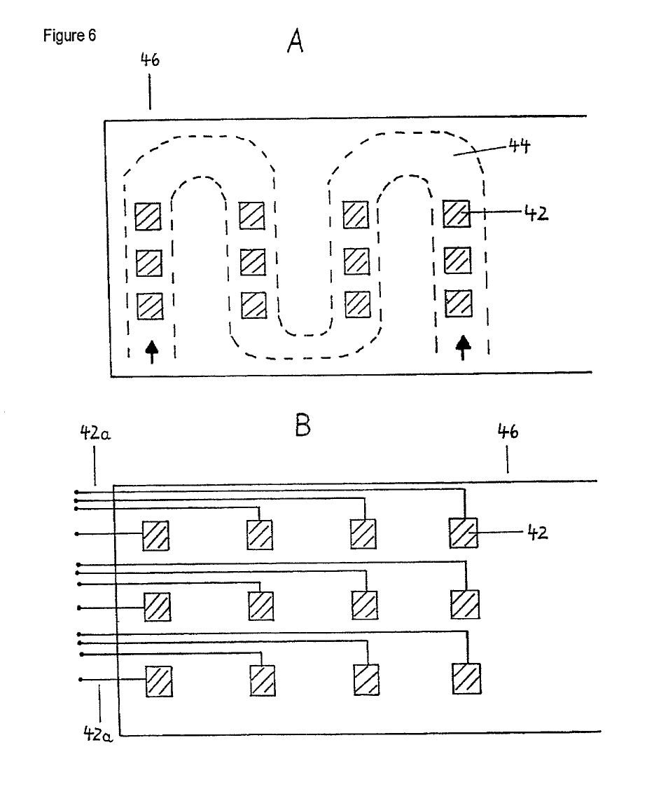

FIG. 6A and FIG. 6B show an alternative electrode structure (42) in connection with a channel structure (44) of the support (46). The electrically conductive connections (42a) shown in FIG. 6B run from the electrodes (42) to an edge of the support.

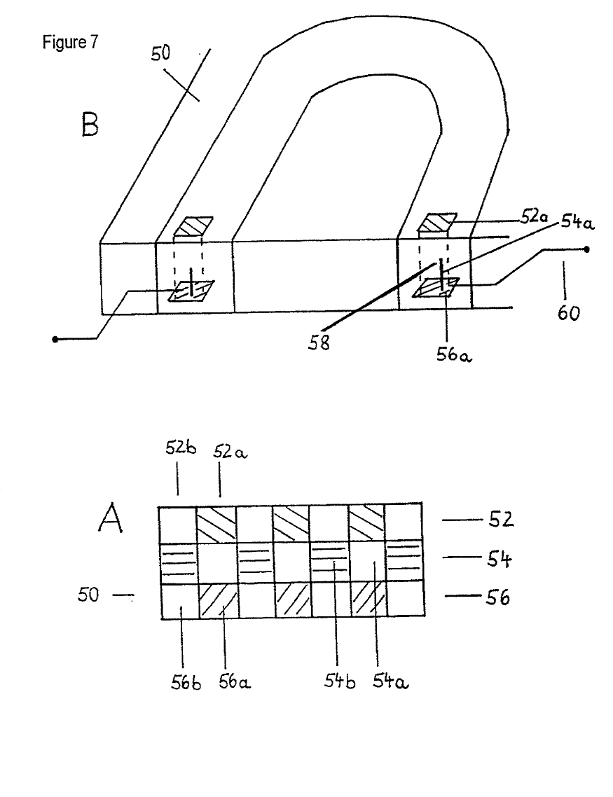

FIG. 7A and FIG. 7B show a projection of the light source matrix through the microfluidic support onto the electronic detection matrix. The support (50) contains a light source matrix (52) with active pixels (52a) and nonactive pixels (52b), a fluidic area (54) with one or more channels (54a) and structural areas of the support matrix (54b) and electronic detection matrix (56) with a plurality of electrodes (56a) and non-electrode areas (56b). Receptors (58) are immobilized on the electrodes. The electrodes furthermore have an electrically conductive connection (60). Active pixels of the light source matrix (52a) and of the electronic detection matrix (56a) are preferably arranged directly above one another.

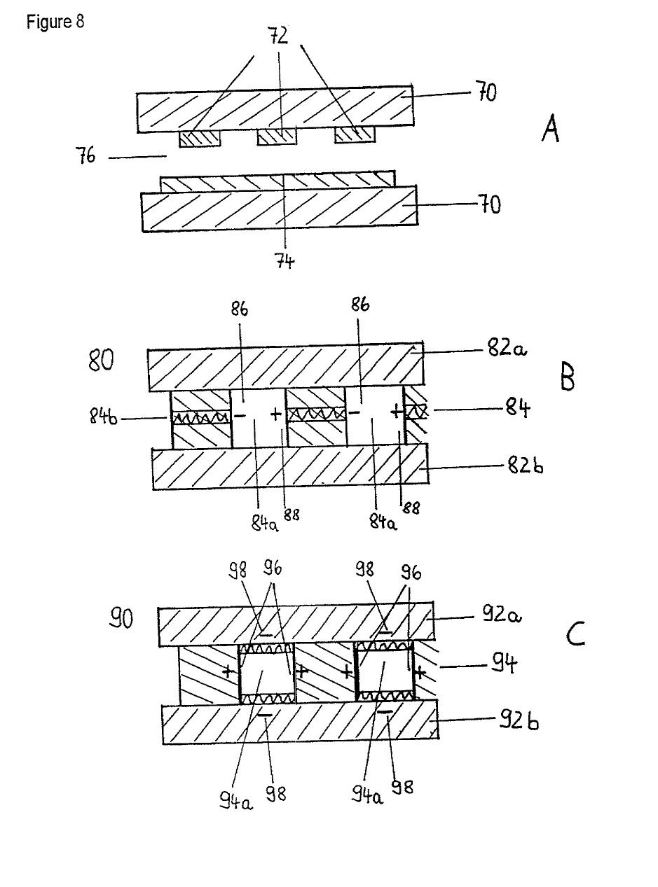

FIG. 8 shows variants of the connection technique for measuring an electronic detection signal, e.g. a glass, e.g. Pyrex/metal, e.g. a silicon/glass, e.g. Pyrex sandwich structure. In the embodiment of the support (70) shown in FIG. 8A, electrodes, preferably transparent electrodes, are arranged in the form of columns (72) and rows (74) on the top and bottom sides of the fluid channel (76).

In the embodiment shown in FIG. 8B, the support structure (80) has a sandwich-like arrangement, with two cover layers (82a, 82b) being arranged above and below, respectively, a structural layer (84) containing the fluidic system. The cover layers (82a, 82b) are preferably, at least in the area of the microchannels (84a), optically transparent, for example made of glass. The intermediate layer (84) consists at least partially of a conductive material, for example of metal, e.g. silicon. Conducting sublayers (84b) which provide the electrodes may be provided on the walls (86, 88) of the structural layer (84) surrounding a microchannel (84a).

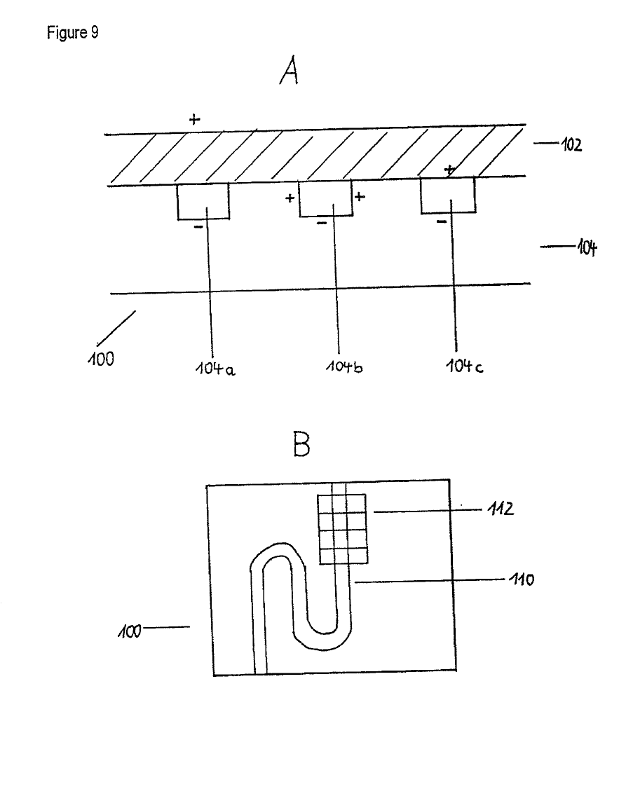

The support structure (90) shown in FIG. 8C is constructed similarly to the support structure according to FIG. 8B. It contains 2, preferably optically transparent, cover layers (92a, 92b) and in between a structured layer (94), for example a metal layer such as, for example, silicon, with microchannels (94a). The walls of the structural layer (94) which are adjacent to the microchannel contain, at least partially, an electrically conductive sublayer (96), for example a positively charged layer. Opposite electrodes, preferably transparent opposite electrodes (98), are arranged on the top or/and bottom side of the microchannel (94).

Whereas the embodiments shown in FIG. 8 are suitable in particular for supports working according to the transmitted-light principle, FIG. 9 shows an embodiment for back light. The support structure (100) contains an optically transparent cover layer (102) through which the light of the light source matrix (not shown) can be introduced and reflected again. Furthermore, a structural layer (104) is provided which preferably consists of metal or another fully or partially conductive material, for example a doped plastic material. The material of the structural layer is particularly preferably silicon. Microchannels (104a, 104b, 104c) are provided in the structural layer (104). In microchannel (104a), an electrode (−) on the bottom of the microchannel and an external opposite pole (+) are provided. In microchannel (104b), an electrode (−) on the bottom and opposite poles (+) on the wall are provided. In microchannel 104c, an electrode (−) on the bottom and an internal opposite pole (+) at the top, for example a transparent electrode as described above, are provided. FIG. 9B is a plan view of the apparatus depicted in FIG. 9A and shows the support structure (100) with the microchannel (110) and electrodes (112) arranged along the microchannel.

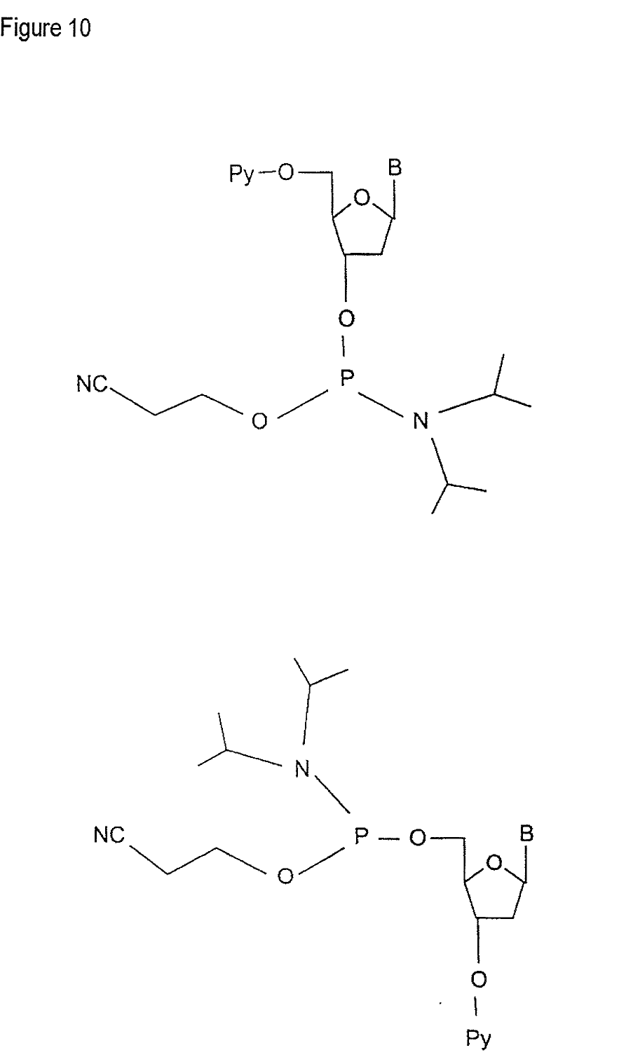

FIG. 10 finally shows preferred nucleotide building blocks for the electronically controlled in situ nucleic acid synthesis. Py is an electronically removable protective group, for example p-nitrobenzyloxycarbonyl, 2-(p-nitrophenyl)ethyl oxycarbonyl, 2,4-dinitrobenzyloxycarbonyl or 2,4-(p-dinitrophenyl)ethyloxycarbonyl.

1. A method for determining analytes, which comprises the following steps:
(a) providing an apparatus comprising
(i) a light source matrix,
(ii) a microfluidic support having channels which contain a plurality of predetermined areas at which in each case different receptors are immobilized on the support,
(iii) means for supplying fluids to the support and for discharging fluids from the support and
(iv) an electronic detection matrix having a plurality of electrodes assigned to the predetermined areas containing immobilized receptors on the support,
(b) contacting the support with a sample containing analytes and
(c) determining the analytes by electronic detection via binding thereof to the receptors immobilized on the support.

2. The method as claimed in claim 1, wherein a programmable light source matrix selected from the group consisting of a light valve matrix, a mirror array and a UV-laser array is used.

3. The method as claimed in claim 1 or 2, wherein a microfluidic support with closed channels is used.

4. The method as claimed in claim 1, wherein an apparatus is used which contains at least the components (ii), (iii) and (iv) in an integrated form.

5. The method as claimed in any of claims 1 to 4, wherein electrodes are used which contain a conductive material such as, for example, a metal, a conductive polymer or a conductive glass.

6. The method as claimed in any of claims 1 to 4, wherein electrodes having an area in the range from 15-250,000 μm² are used.

7. The method as claimed in any of claims 1 to 6, wherein the electronic detection comprises measuring the conductivity, impedance, voltage and/or current via said electrodes.

8. The method as claimed in claim 7, wherein the measurement comprises a potentiometric measurement, a cyclovoltametric measurement, an amperometric measurement or a chronopotentiometric measurement.

9. The method as claimed in any of claims 1 to 8, wherein the detection comprises a light source matrix-initiated redox process which correlates with the binding of analytes to the receptors immobilized on the support.

10. The method as claimed in any of claims 1 to 9, wherein the receptors are selected from biopolymers such as, for example, nucleic acids, nucleic acid analogs, proteins, peptides and carbohydrates.

11. The method as claimed in claim 10, wherein the receptors are selected from the group consisting of nucleic acids and nucleic acid analogs and binding of the analytes is a hybridization.

12. The method as claimed in any of claims 1 to 11, wherein a plurality of analytes are determined in parallel in the sample.

13. The method as claimed in claim 12, wherein at least 50, preferably at least 100, analytes are determined in parallel.

14. The method as claimed in any of claims 1 to 13, wherein the receptors are immobilized to the support via covalent binding, noncovalent self assembly, charge interaction or combinations thereof.

15. The method as claimed in any of claims 1 to 14, wherein the receptors are synthesized in situ on the support.

16. The method as claimed in claim 15, wherein the receptor synthesis comprises:
directing fluid containing receptor synthesis building blocks over the support, immobilizing said building blocks time- and/or location-specifically at in each case predetermined positions on the support and repeating said steps until the desired receptors have been synthesized at the in each case predetermined positions.

17. The method as claimed in claim 15 or 16, wherein the receptor synthesis comprises at least one illumination step initiated by the light source matrix or/and a process step mediated by the electronic detection matrix.

18. The method as claimed in any of claims 15 to 17, wherein receptor synthesis comprises on-line process monitoring.

19. The method as claimed in claim 18, wherein the on-line process monitoring is carried out by the electronic detection matrix.

20. The method as claimed in any of claims 15 to 19, wherein electronically removable protective groups such as, for example, p-nitrobenzyloxycarbonyl or 2,4-dinitrobenzyloxycarbonyl are used for receptor synthesis.

21. An apparatus for determining analytes, which comprises
(i) a light source matrix,
(ii) a support containing a plurality of predetermined positions at which in each case different receptors are immobilized on the support,
(iii) means for supplying fluids to the support and for discharging fluids from the support and
(iv) an electronic detection matrix having a plurality of electrodes assigned to the predetermined positions containing immobilized receptors on the support.

22. The apparatus as claimed in claim 21, wherein at least the components (ii), (iii) and (iv) are present in an integrated form.

23. The apparatus as claimed in either of claims 21 and 22, wherein the support is arranged between light source matrix and electronic detection matrix.

24. The use of an apparatus as claimed in any of claims 21 to 23 in a method for parallel determination of a multiplicity of analytes.
